(19) United States
(12) Patent Application Publication
(10) Pub. No.: US 2024/0010676 A1
(43) Pub. Date: Jan. 11, 2024

(54) METHOD FOR SEPARATION OF PROTEINS FROM PHENOLIC COMPOUNDS

(71) Applicant: Lihme Protein Solutions APS, Farum (DK)

(72) Inventors: Allan Otto Fog Lihme, Farum (DK); Marie Bendix Hansen, Frederiksberg (DK); Bodil Kjær Lindved, Espergærde (DK)

(21) Appl. No.: 18/254,552

(22) PCT Filed: Nov. 24, 2021
(86) PCT No.: PCT/EP2021/082763

§ 371 Date: May 25, 2023

(30) Foreign Application Priority Data
Nov. 27, 2020 (DK) PA 2020 70791

(51) Int. Cl. C07K 1/34; B01D 61/14; B01D 61/58; B01D 61/16; A23J 1/00; A23J 1/14; A23J 1/18; A23K 20/147; A61L 2/10

(52) U.S. Cl. CPC C07K 1/34; B01D 61/147; B01D 61/58; B01D 61/16; A23J 1/007; A23J 1/006; A23J 1/14; A23J 1/18; A23J 1/009; A23K 20/147; A61L 2/10; B01D 2315/10; B01D 2315/16; B01D 2311/08; B01D 2311/04; B01D 2311/06; B01D 2311/02; A61L 2202/21

(57) ABSTRACT

A method for separation of proteins, particularly plant proteins, from salts and phenolic compounds in a liquid comprising said proteins dissolved in said liquid is provided. The method includes a step of subjecting said liquid to a first cross-flow membrane filtration process wherein the first salts and at least a portion of the phenolic compounds migrate across the membrane into a first permeate and the proteins are retained in a first retentate; followed by a step of adding water, or one or more second salts and water to the first retentate, while continuing the membrane filtration process, to create a diafiltrate containing at least a portion of said phenolic compounds and the added second salts.

### TECHNICAL FIELD OF THE INVENTION

The present invention relates to methods for isolating compounds, particularly functional proteins and phenolic compounds, from materials containing such compounds, particularly plant materials, to the isolated compounds, to compositions comprising the isolated compounds and to use and application of the isolated compounds.

### BACKGROUND OF THE INVENTION

Many biological fluids and extracts contain soluble proteins and soluble phenolic compounds such as phenolic acids, flavonoids and tannins. This is particularly the case for liquid plant extracts and extracts of e.g. yeast and algae. Many of the phenolic compounds are highly reactive and may e.g. be oxidized to create oligomers and polymers. Such phenolic compounds may also adsorb to and even react covalently with proteins. The reactivity of phenolic compounds towards adsorption and/or covalent reaction with proteins is influenced by many physico-chemical parameters such as the degree of oxidation, pH, temperature and time of contact.

Compounds such as proteins and metabolites comprised in plants are valuable and useful in many different applications such as nutrition, medical treatments, cosmetics and acceptable process aids for industrial manufacture of the same. Particularly, such proteins and metabolites in significant crop plants are interesting and become even more valuable and useful in isolated form. Grass, and the green leaves of other plants for example, contain useful rubisco protein and phenolic compounds which are desirable to use in isolated and more pure forms. Leaf and grass proteins are potentially the cheapest and most abundant source of protein in the world. They are also highly nutritious and have many desirable functional characteristics which could make them useful in both food and industrial products.

Proteins may be used as techno-functional ingredients in the preparation of foodstuffs, for example to provide gelling, foaming or emulsification, and for these purposes the proteins need to be devoid of compounds that may give rise to unwanted color and taste formation and influence the quality and stability of the prepared food.

Phenolic compounds may themselves constitute valuable products for a variety of applications e.g. as antioxidants, anti-cancer agents and antimicrobial agents.

Phenolic compounds are secondary metabolites produced in plants that have a common structure based on an aromatic ring with one or more hydroxyl substituents. Their presence affects the sensory qualities of plant-derived processed foods, including taste, color, and texture.

The predominant phenolic acids in plants are substituted derivatives of hydroxybenzoic acids and hydroxycinnamic acids. Caffeic, p-coumaric, and ferulic acids are the most common hydroxycinnamic acids and frequently occur in foods as esters with quinic acid or sugars; hydroxybenzoic acid derivatives are mainly present in foods in the glucoside forms, and p-hydroxybenzoic, vanillic, and protocatechuic acids are the most common forms.

Flavonoids represent the most common group of plant phenolic compounds and their presence influences the flavor and color of fruits and vegetables. The six significant subclasses of flavonoids are the flavones, flavonols, flavanones, flavan-3-ols, anthocyanidins, and isoflavones. Occasionally they can be found as aglycones but most flavonoids are attached to sugars (glycosides).

Thus, the separation of soluble proteins and phenolic compounds can be important for obtaining the necessary purity and quality of these valuable compounds.

Membrane filtration, and in particular cross-flow ultrafiltration and diafiltration are well known methods for separation of high molecular weight compounds like proteins and low molecular weight compounds like carbohydrates and minerals (salts). A widespread example of this technique is the manufacture of whey protein concentrates and whey protein isolates from cheese whey by combined ultrafiltration and diafiltration.

Diafiltration is a technique that uses membranes to completely remove, replace, or lower the concentration of salts or other low molecular weight substances from solutions containing proteins, peptides, nucleic acids, and other high molecular weight biomolecules. The process selectively utilizes permeable (porous) membrane filters to separate the components of solutions and suspensions based on their molecular size. An ultrafiltration membrane retains molecules that are larger than the pores of the membrane while smaller molecules such as salts, phenolic compounds, solvents and water, which may pass through the membrane. In a diafiltration process the retentate is added water, while the membrane filtration process continuously removes water, salts and low molecular weight compounds to the permeate side of the membrane.

B. Dutre and G. TrtigMh (1994) Macrosolute-microsolute separation by ultrafiltration: A review of diafiltration processes and applications. Desalination, 95, 227-267 discloses a review of the theory and applications of diafiltration in several commercial scale examples.

However, for certain separation tasks, such as the separation of proteins and phenolic compounds, and in particular the separation of soluble proteins from phenolic compounds in plant extracts, the membrane filtration technology is not a complete and cost-efficient process, which may be due to the tendency of phenolic compounds to adhere to other compounds such as proteins, polysaccharides as well as the membrane surface.

For example, according to several scientific reports (e.g. Straetkvern K O & Schwarz J G (2012) Recovery of Native Potato Protein Comparing Expanded Bed Adsorption and Ultrafiltration. Food and Bioprocess Technology. 5(5), 1939-1949 and Zwijnenberg H J, Kemperman A J B, Boerrigter M E, Lotz M, Dijksterhuis J F, Poulsen P E & Koops G H (2002) Native protein recovery from potato fruit juice by ultrafiltration. Desalination. 144(1-3), 331-334) ultrafiltration has low selectivity and only poorly separate polyphenols and brown polyphenol complexes from proteins thus giving a powder with a final brown hue and higher content of chlorogenic acids, and in addition it is often encountered with membrane concentration of potato fruit juice that fouling of the membranes lead to low flux rates, low system productivity and a short membrane lifetime. Both disclosures describe the use of water for the applied diafiltration step. In Zwijnenberg et al it is illustrated how the permeate flux decrease with the onset of diafiltration using water as the diafiltration solvent.

Benny E. Knuckles, Donald de Fremery, E. M. Bickoff, and George 0. Kohler (1975) Soluble Protein from Alfalfa Juice by Membrane Filtration. J. Agr. Food Chem. Vol. 23, No. 2, 209-212 discloses the separation of protein from pigmented components (phenolic compounds) from Alfalfa juice by ultrafiltration and diafiltration. Diafiltration was performed with water and even when ten times the retentate volume of water was used for the diafiltration the protein solution still contained a significant concentration of low molecular weight and highly pigmented compounds.

EP 2 934 187 B1 discloses the separation of rubisco protein from polyphenols in extracts of sugar beet leaves amongst other steps by the use of ultrafiltration and diafiltration. The disclosure indicates that diafiltration may be performed with water or a non-specified food grade buffer but only 25-75% of the polyphenols may be removed from the protein by this procedure.

Thus, there is a need for improved methods for economic separation of proteins and phenolic compounds at industrial scale.

### SUMMARY OF THE INVENTION

In a first aspect, the present invention relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water, to the retentate while continuing the membrane filtration process to create a diafiltrate containing at least a portion of said phenolic compounds and the added second salts

In a further improved commercial embodiment the present invention combines the first aspect with separation of the phenolic compounds from the one or more first salts present in the first permeate and/or the added second salts in the diafiltrate such that the phenolic compounds may be isolated in a purified state and the salts may be recycled for use in the diafiltration step mentioned above.

Thus, in a second aspect, the present invention further relates to a method for separation of proteins from one or more first salts and phenolic compounds in a liquid comprising said proteins dissolved in said liquid, said method comprising the steps of
i. providing a liquid comprising said proteins dissolved in said liquid, said liquid further comprising one or more first salts and phenolic compounds;
ii. Subjecting said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water, to the retentate while continuing the membrane filtration process to create a diafiltrate containing at least a portion of said phenolic compounds and the added second salts
iv. Subjecting the first permeate and/or said diafiltrate from said first cross-flow membrane filtration process to a second cross-flow membrane filtration process wherein at least a portion of the salts present herein migrate across the membrane into a second permeate and the phenolic compounds are retained in a second retentate

In a third aspect, the present invention further relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water to the retentate while continuing the membrane filtration process to create a diafiltrate containing at least a portion of said phenolic compounds and the added second salts
iv. Subjecting the first retentate to a microfiltration membrane filtration process wherein at least a portion of the proteins soluble in said retentate migrate across the membrane into a microfiltration permeate with a lower turbidity than the turbidity of said first retentate;

A preferred embodiment of the invention combines the important features of the invention into an even further commercially improved separation process.

Thus, in a fourth aspect, the present invention further relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water to the retentate while continuing the membrane filtration process to create a diafiltrate containing at least a portion of said phenolic compounds and the added second salts
iv. Subjecting the first permeate and/or said diafiltrate from said first cross-flow membrane filtration process to a second cross-flow membrane filtration process wherein at least a portion of the salts present herein migrate across the membrane into a second permeate and the phenolic compounds are retained in a second retentate
v. Subjecting the first retentate to a microfiltration membrane filtration process wherein at least a portion of the proteins soluble in said retentate migrate across the membrane into a microfiltration permeate with a lower turbidity than the turbidity of said first retentate

### DETAILED DESCRIPTION

### Definitions

The term “plant protein(s)” means protein or proteins which are naturally present in plants.

The term “functional proteins” means proteins that have a high level of techno functional properties such as the ability to form gels when heated in solution, to create foams when aqueous solutions of the protein are whipped with air or to create emulsions when mixed with lipids in aqueous solutions of the protein.

The term “insoluble fibers” means substances present in the liquid comprising proteins that can be separated from the liquid by centrifugation in a laboratory centrifuge at 4000 rpm for 30 minutes at room temperature. The precise chemical composition of the insoluble fibers may vary broadly, but may typically comprise insoluble polysaccharides, pectinates, starches and proteins and insoluble complexes of one or more of these substances.

The term “patatin”, also denoted herein as “PA”, means storage glycoproteins found in potatoes (Solanum tuberosum). Patatin represents a group of immunologically identical glycoprotein isoforms with molecular mass in the range of 40-43 kDa. Patatin also have phospolipase activity capable of cleaving fatty acids from membrane lipids. For purposes of the invention PA may be determined by different known assays, including SDS-PAGE combined with scanning densitometry as described herein (e.g. using a GS-900™ Calibrated Densitometer from BIO-RAD Laboratories, USA) including all protein bands in the molecular weight region between 35 kD and 60 kD the PA category, ELISA testing using patatin specific antibodies, as well as enzymatic assays specific for the phospholipase activity (see e.g. Lipids, 2003, 38(6):677-82. “Determination of the phospholipase activity of patatin by a continuous spectrophotometric assay.” Jiménez-Atiénzar M et al.

The term “protease inhibitor”, also denoted herein as “PI”, means proteins, which possess molecular weights ranging from about 3 kD to about 35 kD, e.g. found in potatoes (Solanum tuberosum) and other plants such as soy and lupin, animals and microorganisms capable of inhibiting the activity of e.g. serine proteases, cysteine proteases, aspartate proteases, and metalloproteases. For purposes of the invention PI, in e.g. potato derived samples, may be determined by different known assays, including SDS-PAGE combined with scanning densitometry as described herein (e.g. using a GS-900™ Calibrated Densitometer from BIO-RAD Laboratories, USA) including all protein bands in the molecular weight region between 3 kD and 35 kD in the PI category, and more broadly by enzyme inhibition assays as generally described in the art (see e.g. The Open Food Science Journal, 2011, 5:42-46. “Quantitative Determination of Trypsin Inhibitory Activity in Complex Matrices”. Robin E. J. Spelbrink et al.).

The term “polyphenol oxidase”, also denoted herein as “PPO”, means proteins found in nearly all plant tissues including potatoes (Solanum tuberosum), and can also be found in bacteria, animals, and fungi. Polyphenol oxidase (tyrosinase) (TY) is a bifunctional, copper-containing oxidase having both catecholase and cresolase activity. PPO causes the rapid polymerization of o-quinones to produce black, brown or red pigments (polyphenols) which cause fruit browning. The amino acid tyrosine contains a single phenolic ring that may be oxidised by the action of PPOs to form o-quinone. Hence, PPOs may also be referred to as tyrosinases. The catalytic action of PPO has a negative impact on the quality of several fruit and vegetable crops and results in alteration of color, flavor, texture, and nutritional value. It is a limiting factor in the handling and technological processing of crops as peeled, sliced, bruised or diseased tissues rapidly undergo browning. For purposes of the invention PPO may be determined by different known assays as reviewed in: Journal of Food Biochemistry 2003, 27(5):361-422. “Physicochemical properties and function of plant polyphenol oxidase: A review”. Ruhiye Yoruk et al.

The term “lipoxygenase”, also denoted herein as “LipO”, means proteins found in found in plants, animals and fungi capable of catalyzing the dioxygenation of polyunsaturated fatty acids. Lipoxygenases have food-related applications in bread making and aroma production but they also have negative implications for the color, off-flavour and antioxidant status of plant-based foods. In potatoes (Solanum tuberosum) lipoxygenase has a molecular weight of approx. 97 kD and can be detected by SDS-PAGE (see e.g. FEBS Journal, 2006, 273,3569-3584 “Patatins, Kunitz protease inhibitors and other major proteins in tuber of potato cv. Kuras” Guy Bauw et al.). For purposes of the invention LipO may be determined by different known assays, including SDS-PAGE combined with scanning densitometry as described herein (e.g. using a GS-900™ Calibrated Densitometer from BIO-RAD Laboratories, USA) as wells as enzyme activity assays as described in e.g. J. Agric. Food Chem., 2001, 49, 32-37. “Colorimetric Method for the Determination of Lipoxygenase Activity”. Gordon E. Anthon et al.

The term “glycoalkaloid” or “alkaloid glucoside” means a family of chemical compounds derived from alkaloids in which sugar groups are appended. There are several that are potentially toxic, most notably those which are the poisons commonly found in the plant species Solanum dulcamara (nightshade). A prototypical glycoalkaloid is solanine (composed of the sugar solanose and the alkaloid solanidine), which is found in potatoes (Solanum tuberosum). For purposes of the invention glycoalkaloid may be determined by different known assays, including a standard HPLC assay as described Eng. Life Sci., 2005, 5, 562-567. “Optimization of glycoalkaloid analysis for us in industrial potato fruit juice downstreaming”. Alt, V., Steinhof et al.

The term “first salts” means salts that are present in the liquid at the outset of the separation process (and includes “natural salts”, being the salts which are naturally present in plant, yeast or algae).

The term “second salts” means the one or more salts added with water during the diafiltration step

The term “phenolic compounds” means aromatic or heteroaromatic compounds comprising one or more ring systems and one or more phenolic hydroxyl groups. Plant phenolic compounds include phenolics acids, flavonoids, tannins, stilbenes and lignans

The term “protein concentration” means the amount of protein per liter of a sample calculated as the total weight or mass of amino acids per liter as determined according to EUROPEAN PHARMACOPOEIA 5.0 section 2.2.56. AMINO ACID ANALYSIS or by determination of total nitrogen in a sample by the method of Kjeldahl using the conversion factor N×6.25. All samples are dialyzed against demineralized water in dialysis tubing cellulose membrane (Sigma-Aldrich, USA, cat. No.: D9652) to remove any free amino acids and low molecular weight peptides prior to the amino acid determination.

The term “dry weight” means the weight or mass of a substance remaining after removal of water by heating to constant weight at 110 degrees Celcius. The dry weight per ml sample is thus the weight or mass of a substance remaining after removal of water by heating to constant weight at 110 degrees Celcius per ml sample applied to drying.

The term “absorbance at 600 nm” means an expression of the amount of light of wavelength 600 nm passing through a liquid sample when measured in a spectrophotometer using 10 mm light path cuvettes. The absorbance of a liquid sample is often expressed as O.D. 600 nm. For the purpose of this disclosure the absorbance is determined by spectrophotometry on samples diluted in 0.05 M potassium phosphate pH 7.0 to a read out in the range of 0.5 to 1.0 and the read out is multiplied with the dilution factor to determine the absorbance of the undiluted sample. If a sample has an absorbance of less than 1.0 no dilution is performed, but pH must be adjusted to 7.0 with sodium hydroxide or hydrochloric acid.

The term “soluble” means solubility in water at a concentration of at least 1 g/L at 25 degrees Celsius.

The term “pectin” means pectic polysaccharides, which are rich in galacturonic acid. The amount, structure and chemical composition of pectin differs among plants, within a plant over time, and in various parts of a plant. In natural pectins around 80 percent of carboxyl groups of galacturonic acid are esterified with methanol or are acetylated.

The term “diafiltration” means a technique that uses membranes to completely remove, replace, or lower the concentration of salts or other low molecular weight substances from solutions containing proteins, peptides, nucleic acids, and other high molecular weight molecules. The process selectively utilizes permeable (porous) membrane filters to separate the components of solutions and suspensions based on their molecular size. An ultrafiltration membrane retains molecules that are larger than the pores of the membrane while smaller molecules such as salts, phenols, solvents and water, which may pass through the membrane. In a diafiltration process the retentate is added water or a buffer composition while the membrane filtration process continuously removes water, salts and low molecular weight compounds to the permeate side of the membrane.

The term “nanofiltration” means a membrane filtration-based method that uses nanometer sized through-pores that pass through the membrane. Nanofiltration membranes typically have pore sizes from 1-10 nanometers, smaller than that used in microfiltration and ultrafiltration, but just larger than that in reverse osmosis. Nanofiltration membranes are defined by the molecular weight cut-off (MWCO) of the membrane used. Nanofiltration is applied in cross-flow mode under increased pressure.

The term “ultrafiltration” means a variety of membrane filtration processes in which forces like pressure or concentration gradients lead to a separation through a semipermeable membrane. Suspended solids and solutes of high molecular weight are retained in the so-called retentate, while water and low molecular weight solutes pass through the membrane in the permeate (filtrate). This separation process is typically used in industry and research for purifying and concentrating macromolecular solutions, especially protein solutions. Ultrafiltration membranes are defined by the molecular weight cut-off (MWCO) of the membrane used. Ultrafiltration is applied in cross-flow or dead-end mode. In the context of this disclosure the term “ultrafiltration” includes the meaning of the term “dialysis”

The term “dialysis” means the separation of large molecules, such as proteins, from small molecules and ions in a solution by allowing the latter to pass through a semipermeable membrane by diffusion driven by concentration differences between the liquid phases on each side of the membrane.

The term “microfiltration” means a separation technique for removing micron-sized particles, like bacteria, suspended solids and colloid particles from liquid solutions. The process uses membrane filters with pores in the approximate size range 0.1 to 10 μm, which are permeable to the fluid and dissolved substances, but retain the particles, thus causing separation. Microfiltration membranes are defined by the nominal pore size of the membrane. Microfiltration is applied in cross-flow or dead-end mode.

The term “flux” means the flow of liquid permeating the membrane in a membrane filtration process as described herein measured and expressed in liter permeate per hour per square-meter of membrane area employed.

The term “turbidity” means the measure of relative clarity of a liquid. It is an optical characteristic of water and is a measurement of the amount of light that is scattered by material in the water when a light is shined through the water sample. The higher the intensity of scattered light, the higher the turbidity. Material that causes water to be turbid include clay, silt, very tiny particles of inorganic and organic matter, colloid fibers. Turbidity may be measured with a nephelometer. The units of turbidity from a calibrated nephelometer are called Nephelometric Turbidity Units (NTU).

The term “mineral acids” means the acids hydrochloric acid, nitric acid, phosphoric acid, sulfuric acid.

The term “comprise” and “include” as used throughout the specification and the accompanying aspects and claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

Method for Separation of Proteins from Salts and Phenolic Compounds

The present invention improves the commercial and industrial applicability of membrane filtration for separation of proteins and phenolic compounds by the use of diafiltration solvents with an increased ionic strength and pH control which leads to a higher permeability of the phenolic compounds and a higher average permeate flux during the diafiltration step.

Thus, in a first aspect, the present invention relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water to the retentate while continuing the membrane filtration process to create a diafiltrate containing at least a portion of said phenolic compounds and the added second salts

In a further improved commercial embodiment the present invention combines the first aspect with a further process step separating the phenolic compounds from the one or more first salts present in the first permeate and/or the added salts in the diafiltrate such that the phenolic compounds may be isolated in a purified state and thereby become a product of higher value. And as a further highly important cost saving step the salts thus separated from the phenolic compounds may then be recycled for use in the diafiltration step mentioned in iii. above. By doing so there is not only saved a significant amount of water and salts for the process but there will also be cost savings related to disposure of waste. In an aspect of the invention the addition of one or more added salts and water of step iii. is fully, or predominantly, performed using the one or more first salts and water separated from phenolic compounds in said further process step.

Thus, in a second aspect, the present invention further relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid comprising proteins dissolved in said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water to the retentate while continuing the membrane filtration process to create a diafiltrate containing at least a portion of said phenolic compounds and the added second salts
iv. Subjecting the first permeate and/or said diafiltrate from said first cross-flow membrane filtration process to a second cross-flow membrane filtration process wherein at least a portion of the salts present herein migrate across the membrane into a second permeate and the phenolic compounds are retained in a second retentate

In a preferred embodiment said second permeate is used, at least in part, as the source of said one or more second salts and water in step iii. In an aspect of the invention the addition of one or more added salts and water of step iii. is fully, or predominantly, performed using the one or more first salts and water present in the first permeate and separated from phenolic compounds in step iv.

For certain applications it is preferred that the liquid comprising the separated proteins has a low level of turbidity and it may therefore be relevant to perform a further separation step to remove suspended or colloid particles in the liquid. Centrifugation is a very well-known method for separation of suspended solids from liquid solutions. However, potato fruit juice or similar vegetable juices may be extremely difficult and/or expensive to clarify by centrifugation methods at large scale. Microfiltration is an alternative method for clarification but also this method has serious shortcomings due to fouling of the membrane surface with low productivity and low product permeability as the result. Therefore, it is not possible to efficiently apply microfiltration on crude vegetable juice without serious loss of product and long processing times.

It has now surprisingly been found that the microfiltration process show significantly less tendency to fouling when the liquid to be filtered has already been separated from phenolic compounds that otherwise tend to adsorb to the surface of the membrane and block the pores.

Thus, in a third aspect, the present invention further relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said liquid comprising proteins dissolved in said liquid to a first cross-flow membrane filtration process wherein the one or more first salts and at least a portion of the phenolic compounds migrate across the membrane into a first permeate and the proteins are retained in a first retentate;
iii. Adding water, or one or more second salts and water, to the retentate while continuing the membrane filtration process to create a diafiltrate containing at least a portion of said phenolic compounds and the added second salts
iv. Subjecting the first retentate to a microfiltration membrane filtration process wherein at least a portion of the proteins soluble in said retentate migrate across the membrane into a microfiltration permeate with a lower turbidity than the turbidity of said first retentate;

In an aspect of the invention the microfiltration permeate of step iv. is further treated with a cross-flow membrane filtration process wherein the protein is concentrated in the retentate and the salts are migrating through the membrane to create a third permeate. In a further aspect the third permeate is used as a diafiltration liquid to be added during the microfiltration process of step iv. to wash out further proteins from the retentate into the microfiltration permeate.

A preferred embodiment of the invention combines the important features of the invention into an even further commercially improved separation process.

Thus, in a fourth aspect, the present invention further relates to a method for separation of proteins from one or more first salts and phenolic compounds in a liquid comprising proteins dissolved in said liquid, said method comprising the steps of
i. providing a liquid comprising said proteins dissolved in said liquid, said liquid further comprising one or more first salts and phenolic compounds;
ii. Subjecting said a liquid comprising proteins dissolved in said liquid or a derivative thereof to a first cross-flow membrane filtration process wherein the one or more first salts and at least a portion of the phenolic compounds migrate across the membrane into a first permeate and the proteins are retained in a first retentate;
iii. Adding water, or one or more second salts and water, to the retentate while continuing the membrane filtration process to create a diafiltrate containing at least a portion of said phenolic compounds and the added second salts
iv. Subjecting the first permeate and/or said diafiltrate from said first cross-flow membrane filtration process to a second cross-flow membrane filtration process wherein at least a portion of the salts present herein migrate across the membrane into a second permeate and the phenolic compounds are retained in a second retentate
v. Subjecting the first retentate to a microfiltration membrane filtration process wherein at least a portion of the proteins soluble in said retentate migrate across the membrane into a microfiltration permeate with a lower turbidity than the turbidity of said first retentate

In a preferred embodiment said second permeate is used, at least in part, as the source of said one or more second salts and water in step iii. In an aspect of the invention the addition of one or more added salts and water of step iii. is fully, or predominantly, performed using the one or more first salts and water present in the first permeate and separated from phenolic compounds in step iv.

In an aspect of the invention the microfiltration permeate of step iv. is further treated with a cross-flow membrane filtration process wherein the protein is concentrated in the retentate and the salts are migrating through the membrane to create a third permeate. In a further aspect the third permeate is used as a diafiltration liquid to be added during the microfiltration process of step iv. to wash out further proteins from the retentate into the microfiltration permeate.

Liquids Comprising Proteins, Salts and Phenolic Compounds

The proteins to be separated according to the invention are selected from the group of plant proteins, yeast proteins and algae proteins.

In one aspect of the invention the phenolic compounds to be separated according to the invention comprise flavonoids including flavonols, flavanols, and anthocyanins.

In one aspect of the invention the phenolic compounds comprise chlorogenic acids.

The liquids comprising such proteins may be obtained by disintegration, e.g. by grinding, shredding and/or pressing, of the raw materials whereby an aqueous solution comprising protein is released as a juice, and/or the components may be extracted by addition of water or an aqueous extractant solution in combination with physical disruption of the plant tissue and/or cells. Antioxidants, such as sodium sulfite, sodium metabisulfite and ascorbic acid may preferably be added prior to or during the procedures performed to produce the liquid comprising proteins. Likewise, it may be preferable to add salts to control conductivity and pH controlling substances such as acids or bases to adjust pH to a preferred value.

In a preferred embodiment the conductivity of the liquid is in the range of 1 to 50 mS/cm, such as a conductivity in the range of 2 to 40 mS/cm, such as a conductivity in the range of 3 to 30 mS/cm, such as a conductivity in the range of 3 to 25 mS/cm, such as a conductivity in the range of 3 to 20 mS/cm, such as a conductivity in the range of 5 to 17 mS/cm.

In a preferred embodiment the pH of the liquid is within the range of pH 2 to pH 10, such as a pH in the range of pH 3.0 to pH 9, such as a pH in the range of pH 3.5 to pH 8.5, such as a pH in the range of pH 4.0 to pH 8.5, such as a pH in the range of pH 4.5 to pH 8.5, such as a pH in the range of pH 5.2 to pH 7.5. For certain applications the pH is preferably in the range of pH 4.2 to pH 6.5, such as a pH in the range of pH 4.5 to pH 6.5, such as a pH in the range of pH 4.8 to pH 6.5, such as a pH in the range of pH 5.0 to pH 6.5.

In a preferred embodiment the liquid has a temperature in the range of 1 to 60° C., such as 5 to 55° C., such as 10 to 50° C., such as 15 to 48° C., such as 12 to 45° C., such as 18 to 30° C., such as 22 to 28° C.

The liquid so obtained may be pretreated in various ways prior to separation according to the invention. Preferably the liquid is pretreated to remove insoluble material of a particle size larger than 10 micron which may be achieved e.g. by centrifugation and/or filtration.

Practically all liquid extracts and juices prepared from plant materials, yeast and algae will contain phenolic compounds such as phenolic acids, flavonoids, tannins, stilbenes, lignans, gallic acid and catechin. Phenolic compounds constitute one of the most numerous and widespread groups of secondary metabolites. These components are important to the normal growth and development of algae and terrestrial plants, providing defense mechanisms against infections, injuries, and environmental aggressions. As part of both animal and human diet, the nutraceutical properties assigned to phenolic compounds are almost endless. Therefore, in recent years, there has been an outstanding demand for the search for phenolic compounds from natural sources, with a focus on plants, fruits, or ensuing agro-industrial biomass residues. Marine macroalgae (seaweeds) have been seen in the last years as a valuable source of bioactive components, including phenolic compounds, which in some cases are exclusive to macroalgae, e.g., phlorotannins.

Most extracts of plants, yeast and algae will contain insoluble fibers, also known as dietary fibers. Such dietary fibers may have health promoting effects. In a preferred embodiment of the invention the liquid comprising proteins, salts and phenolic compounds also contains insoluble dietary fibers at a concentration in the range of 0.1 to 30 mg/ml based on the dry weight of the fiber, such as 0.2 to 20 mg/ml, such as 0.3 to 15 mg/ml, such as 0.5 to 10 mg/ml, such as 1.0 to 5 mg/ml based on the dry weight of the fiber.

In an embodiment of the invention more than 80% of said insoluble fibers in the liquid comprising proteins are small enough to pass a 50 micron filter, such as a 40 micron filter, such as a 30 micro filter, such as a 20 micron filter, such as a 10 micro filter, such as a 5 micron filter.

In an embodiment of the invention said insoluble fibers are present in the liquid comprising protein in a concentration that results in a pellet when the liquid is centrifuged in a tabletop centrifuge at 4000 G for 30 min. In an embodiment of the invention said pellet constitutes in the range of 0.1 to 10 vol/vol %, such as 0.1 to 5 vol/vol %, such as 0.1 to 3 vol/vol %, such as 0.1 to 2 vol/vol %, such as 0.1 to 1 vol/vol %, such as 0.2 to 5 vol/vol %, such as 0.2 to 3 vol/vol %, such as 0.3 to 5 vol/vol %, such as 0.3 to 3 vol/vol %, such as 0.4 to 5 vol/vol %, such as 0.4 to 4 vol/vol %, such as 0.5 to 5 vol/vol %, such as 0.5 to 4 vol/vol %, such as 0.5 to 3 vol/vol %, when the potato fruit juice at a pH of pH 6 to pH 7 has been diluted (or concentrated) to a total true protein concentration of 5 mg/ml.

In one aspect, the protein is a protein from plants, yeast or algae, preferably a plant protein.

In one aspect of the invention the liquid is an extract or juice produced from a plant material.

In one aspect of the invention the plant material is a grass or green leaves e.g. from an agricultural crop such as clover, alfalfa, spinach and sugar beets.

In one aspect of the invention the plant material is a tuber, a peel, a pod, a seed or a fruit.

In one aspect the plant material is a legume

In one aspect the plant material is yellow or green peas. In one aspect the plant material is pea pods or pea peels.

In one aspect the plant material is mung beans

In one aspect the plant material is an oilseed

In one aspect the plant material is rapeseeds, including de-oiled rapeseeds. In one aspect the plant material is rapeseed press cake.

In one aspect the plant material is tomato seeds.

In one aspect the plant material is sunflower seeds

In one aspect the plant material is a pulse

In one aspect the plant material is a bean, such as soy bean, fava bean, common bean, castor bean, broad bean.

In one aspect the plant material is chickpeas

In one aspect the plant material is a lupin

In one aspect the plant material is a fungus.

In one aspect the plant material is a mushroom

In one aspect of the invention the liquid is an extract or juice produced from a yeast.

In one aspect the yeast is Saccharomyces cerevisiae

Aquatic plants represent a further preferred source of raw materials for the liquid protein solutions of the invention.

Spirulina is a filamentous, helical, photosynthetic cyanobacteria naturally inhabiting alkaline brackish and saline waters in tropical and subtropical regions. Biochemical analysis has revealed its exceptional nutritive properties, so it is referred in the literature as “super food” or “food of the future”. Spirulina is one of the richest natural sources of proteins and essential amino acids, as well as an excellent source of vitamins (primarily A, K, and vitamin B complex), macro- and micro-elements (calcium, potassium, magnesium, iron, iodine, selenium, chromium, zinc, and manganese), essential fatty acids, including γ-linoleic acid (GLA), glycolipids, lipopolysaccharides, and sulfolipids. Spirulina is especially rich in a variety of pigments, such as chlorophylls, β-carotene, xanthophylls, and phycobilins phycobiliproteins).

In one aspect of the invention the liquid is an extract or juice produced from a cyanobacteria, preferably Spirulina platensis and/or Spirulina maxima.

Duckweed is an aquatic plant of the Lemna family and is particularly rich in proteins. Duckweed is small green freshwater plants with fronds from 1 to 12 mm in diameter. They are the smallest and simplest flowering plant and have one of the fastest production rates with doubling time of 2 to 3 days only. This is because all the plant consists of metabolic active cells with very little structural fiber. Some of the specific properties of duckweed are that the plants have the capability of converting degradable pollutants directly into protein rich fodder.

In one aspect of the invention the liquid is an extract or juice produced from an aquatic plant such as duckweed.

The liquid is preferably water. In one aspect, the first salt(s), proteins and phenolic compounds are all dissolved in said liquid.

In one aspect of the invention the liquid comprising protein is the supernatant obtained after precipitation of substantially other proteins at their isoelectric pH and subsequent centrifugation and/or filtration. In one aspect of the invention the isoelectric precipitation is performed in the range of pH 2.0 to pH 7.0, such as in the range of pH 3.0 to pH 6.8, such as in the range of pH 4.0 to pH 6.6, such as in the range of pH 4.1 to pH 6.4, such as in the range of pH 4.2 to pH 6.3, such as in the range of pH 4.0 to pH 4.8, such as in the range of pH 4.9 to pH 6.3, such as in the range of pH 5.0 to pH 6.2, such in the range of pH 5.1 to pH 6.1, such as in the range of pH 5.5 to pH 6.5.

Cross-Flow Membrane Process and Diafiltration

The cross-flow membrane filtration process to provide the first permeate and the first retentate according to step ii. of the invention is an ultrafiltration process wherein high molecular weight compounds, such as proteins, are retained in the retentate and low molecular weight substances, such as salts and phenolic compounds are migrating through the membrane as a permeate. Preferably the pore size of the ultrafiltration membrane is in the range of 5 to 500 kD, such as 10 to 500 kD, such as 10 to 200 kD, such as 10 to 100 kD, such as 10 to 50 kD, such as 30 to 500 kD, such as 50 to 500 kD. The membrane material may be any kind of membrane suitable for cross-flow filtration, examples of which are polysulfone, poyethersulfone, polyvinyldifluoride, polyacrylonitrile, cellulose acetate and ceramic membranes. Preferably the membrane material is regenerated cellulose, polypropylene or polyethylene.

In an embodiment of the invention the membrane is a flat sheet or a spiral wound flat sheet membrane.

In an embodiment of the invention the membrane is a hollow fiber or tubular membrane. In an embodiment of the invention the hollow fiber membrane has an internal diameter in the range of 0.5 to 3.0 mm, such as in the range of 0.6 to 2.5 mm, such as in the range of 0.7 to 2.2 mm, such as in the range of 0.8 to 2.0 mm, such as in the range of 0.9 to 1.8 mm, such as in the range of 1.0 to 1.7 mm, such as in the range of 1.1 to 1.6 mm.

In an embodiment of the invention the hollow fiber membrane has a length in the range of 30 to 150 cm, such as in the range of 40 to 140 cm, such as in the range of 50 to 130 cm, such as in the range of 60 to 120 cm, such as in the range of 70 to 115 cm, such as in the range of 80 to 110 cm, such as in the range of 90 to 110 cm. In a preferred embodiment the length of the hollow fiber membrane is in the range of 40 to 90 cm, such as in the range of 40 to 80 cm, such as in the range of 50 to 80 cm.

In an embodiment of the invention the hollow fiber membrane material comprise polysulfone, polyethersulfone or permanently hydrophilized polysulfone or polyethersulfone.

In an embodiment of the invention the hollow fiber membrane has a pore size in the range of 5 kD to 500 kD, such as in the range of 10 kD to 200 kD, such as in the range of 20 kD to 100 kD, such as in the range of 30 kD to 75 kD.

In an embodiment of the invention the hollow fiber membrane is an inside-out membrane wherein the transmembrane transport of liquid is performed from the inner lumen of the membrane to the outside surface of the membrane.

In order to optimize the processing logistics, and for some applications the processability of said first retentate, the retentate may advantageously be stored for several hours before the diafiltration of step iii is carried out. Thus, in an aspect of the invention said first retentate is stored for at least 3 hours, such as at least 6 hours, such as at least 12 hours, such as at least 18 hours, such as at least 24 hours, such as at least 36 hours, such as at least 48 hours before the diafiltration step of iii is performed. In a further aspect the diafiltration step iii may be partly carried out before the storage of said first retentate. In yet a further aspect said first retentate may be treated with a preliminary diafiltration step using e.g. water or a low conductivity aqueous solution prior to storage and subsequent diafiltration according to step iii.

In an aspect of the invention the storage temperature of said first retentate is within the range of 1 to 25° C., such as 2 to 20° C., such as 3 to 15° C., such as 2 to 12° C., such as 2 to 10° C., such as 2 to 8° C.

In a further aspect the pH of said first retentate during storage is adjusted to a pH in the range of pH 4 to pH 6.0, such as pH 4.2 to pH 5.8, such as pH 4.5 to pH 5.6, such as pH 4.5 to pH 5.4, such as pH 4.5 to pH 5.2, such as pH 4.7 to pH 5.3.

In an aspect of the invention the volume concentration factor of step ii is in the range of 0.1 to 20, such as in the range of 0.5 to 18, such as in the range of 2.0 to 17, such as in the range of 2.5 to 16, such as in the range of 3.0 to 15, such as in the range of 3.5 to 14, such as in the range of 4.0 to 13, such as in the range of 4.5 to 12, such as in the range of 5.0 to 11, such as in the range of 5.0 to 10.

In an aspect of the invention the true protein concentration of the first retentate is in the range of 5 g/L to range g/L, such as in the range of 8 g/L to 180 g/L, such as in the range of 10 g/L to 160 g/L, such as in the range of 12 g/L to 150 g/L, such as in the range of 15 g/L to 150 g/L, such as in the range of 20 g/L to 150 g/L, such as in the range of 25 g/L to 150 g/L, such as in the range of 30 g/L to 150 g/L, such as in the range of 20 g/L to 140 g/L, such as in the range of 20 g/L to 130 g/L, such as in the range of 20 g/L to 120 g/L, such as in the range of 20 g/L to 100 g/L, such as in the range of 20 g/L to 80 g/L, such as in the range of 20 g/L to 60 g/L, such as in the range of 30 g/L to 60 g/L

Diafiltration Step iii.

In an embodiment of the invention the diafiltration step iii. is performed with the same type of membrane as applied for the ultrafiltration step ii.

The salts to be added in step iii of the invention may be any water soluble salt between one or more of sodium, potassium, ammonium, calcium and magnesium and one or more of the mineral acids or an organic acid. Examples of organic acids are formic acid, acetic acid, propanoic acid, citric acid, caprylic acid, lactic acid, gluconic acid, tartaric acid, fumaric acid, butanedioic acid, benzoic acid.

We have surprisingly found that the diafiltration flux obtained in step iii of the invention may be significantly increased by the addition of calcium and/or magnesium salts. Thus in a particularly preferred embodiment the salts to be added in step iii of the invention is a water soluble calcium or magnesium salt or a mixture of these. Particularly preferred is calcium chloride.

In order to achieve the highest permeability of the salt through a nanofiltration membrane retaining the phenolic compounds in the retentate the one or more salts are preferably chosen from the group of salts constituted by only monovalent inorganic anions and cations, such as sodium chloride, potassium chloride, ammonium chloride.

However, in certain applications there will be a need to limit process costs and the cost of environmentally friendly waste handling. Likewise, certain factory designs cannot withstand the corrosive nature of e.g. chlorides. For such applications of the invention the one or more salts are preferably chosen from the group of sodium sulfate and potassium sulfate.

During diafiltration the salts may be added to the retentate as an aqueous solution or as a solid to be solubilized in the retentate.

The amount of salts added determines the conductivity of the retentate during the diafiltration step. In an aspect of the invention the conductivity of the retentate during the diafiltration step remains within the range of 1 to 50 mS/cm, such as a conductivity in the range of 2 to 40 mS/cm, such as a conductivity in the range of 3 to 30 mS/cm, such as a conductivity in the range of 3 to 25 mS/cm, such as a conductivity in the range of 3 to 20 mS/cm, such as a conductivity in the range of 5 to 17 mS/cm, such as a conductivity in the range of 6 to 25 mS/cm, such as a conductivity in the range of 7 to 25 mS/cm, such as a conductivity in the range of 10 to 25 mS/cm.

The nature of the one or more salts added during the diafiltration step may also influence the pH of the retentate. In a preferred embodiment the pH of the retentate remains within the range of pH 2 to pH 10, such as a pH in the range of pH 3.0 to pH 9, such as a pH in the range of pH 3.5 to pH 8.5, such as a pH in the range of pH 4.0 to pH 8.5, such as a pH in the range of pH 4.5 to pH 8.5, such as a pH in the range of pH 5.2 to pH 7.5. For certain applications the pH is preferably in the range of pH 4.2 to pH 6.5, such as a pH in the range of pH 4.5 to pH 6.5, such as a pH in the range of pH 4.8 to pH 6.5, such as a pH in the range of pH 5.0 to pH 6.5.

When a calcium and/or magnesium salt is added to the retentate prior or during the diafiltration step iii according to the invention it is preferred at the pH of the retentate remains within the range of pH 2 to pH 4.5, such as within the range of pH 2.5 to pH 4.2, such as in the range of pH 2.9 to pH 4.0, such as in the range of pH 3.0 to pH 3.7.

In a preferred embodiment the retentate remains with a temperature in the range of 1 to 60° C., such as 5 to 55° C., such as 10 to 50° C., such as 15 to 48° C., such as 12 to 45° C., such as 18 to 30° C., such as 22 to 28° C.

In a particular aspect of the invention the diafiltration is performed first at a relatively low temperature to remove the majority of the phenolic compounds followed by diafiltration at a relatively higher temperature to remove the remaining phenolic compounds. Thus, in a preferred embodiment the retentate in a first phase of diafiltration remains with a temperature in the range of 1 to 30° C., such as 5 to 28° C., such as 10 to 25° C., such as 15 to 22° C. followed by a second phase of diafiltration wherein the retentate remains with a temperature in the range of 30 to 60° C., such as 30 to 55° C., such as 35 to 52° C., such as 38 to 50° C., such as 40 to 48° C.

The addition of water, or one or more salts and water in step iii. is to be performed enough volume has been added, and subsequently removed in the permeate, to reach a satisfactory lower level of phenolic compounds in the retentate. In an aspect of the invention the target value for remaining phenolic compounds in the retentate corresponds to less than 5000 mg phenolic compounds per kg protein on the basis of dry weight, such as less than 4000 mg/kg, such as less than 3000 mg/kg, such as less than 2000 mg/kg, such as less than 1500 mg/kg, such as less than 1250 mg/kg, such as less than 1000 mg/kg, such as less than 750 mg/kg, such as less than 500 mg/kg, such as less than 200 mg phenolic compounds/kg protein on the basis of dry weight.

In some applications it is further important to reach a lower level of phenolic compounds having a molecular weight below 10 kD. Thus, in a further aspect of the invention the target value for phenolic compounds having a molecular weight below 10 kD remaining in the retentate corresponds to less than 1500 mg/kg, such as less than 1000 mg/kg, such as less than 750 mg/kg, such as less than 500 mg/kg, such as less than 300 mg/kg, such as less than 200 mg/kg, such as less than 100 mg/kg, such as less than 50 mg phenolic compounds/kg protein on the basis of dry weight.

Solanine and chaconine are the major glycoalkaloids present in the nightshade family, which include potato, tomato and eggplant and constitute up to approx. 95% of the total glycoalkaloid content of the potato. Glycoalkaloids are toxic to humans and may at elevated levels be responsible for flavours described as bitter, burning, scratchy or acrid.

In an aspect of the invention also glycoalkaloids are efficiently separated from the proteins present in the liquid. Thus, in an aspect of the invention the diafiltration step iii. brings the total glycoalkaloid remaining in the retentate below a target amount corresponding to less than 500 mg/kg, such as less than 300 mg/kg, such as less than 200 mg/kg, such as less than 100 mg/kg, such as less than 50 mg total glycoalkaloids/kg protein on the basis of dry weight.

In an aspect of the invention the addition of water, or one or more salts and water in step iii. is performed continuously and with practically the same flow rate as the permeate is migrating through the membrane.

In a further aspect the addition of one or more salts and water in step iii is done in small portions, such as portions approximately equal to the retentate volume.

In an aspect of the invention the total volume of water added during the diafiltration step iii. is in the range of 4 to 20 times the volume of the retentate, such as 4 to 15 times, such as 5 to 12 times, such as 5 to 10 times, such as 6 to 10 times, such as 7 to 9 times, such as 5 to 9 times the volume of the retentate.

In a particular aspect of the invention, step iii. comprises a second phase of diafiltration following the addition of one or more salts and water wherein the diafiltration is continued with the addition of water without adding any further salts. In this way the salt concentration and conductivity of the retentate will gradually decrease until a target value for the conductivity has been reached.

In an aspect of the invention, step iii is continued with water and without the addition of further salts until the conductivity of the retentate is less than 10 mS/cm, such as less than 8 mS/cm, such as less than 5 mS/cm, such as less than 4 mS/cm, such as less than 3 mS/cm, such as less than 2 mS/cm, such as less than 1 mS/cm.

In an aspect of the invention the diafiltration step iii is performed entirely without the addition of any second salts such that the diafiltration is performed with water. In an aspect of the invention the diafiltration water is tap water, such as filtered tap water. In an aspect of the invention the diafiltration water is demineralized water. In an aspect of the invention the diafiltration water is recirculated water produced by e.g. reverse osmosis or evaporation of the first permeate of step ii and/or the diafiltration step iii and/or the second permeate of step iv.

In order to optimize the processing logistics, and for some applications the processability of the retentate resulting after the diafiltration step iii, the retentate may advantageously be stored for several hours before further processing, such as the microfiltration step v., drying or further separation steps are carried out.

Thus, in an aspect of the invention the retentate resulting after the diafiltration step iii is stored for at least 3 hours, such as at least 6 hours, such as at least 12 hours, such as at least 18 hours, such as at least 24 hours, such as at least 36 hours, such as at least 48 hours before further processing is performed.

In an aspect of the invention the storage temperature of said retentate resulting after the diafiltration step iii is within the range of 1 to 25° C., such as 2 to 20° C., such as 3 to 15° C., such as 2 to 12° C., such as 2 to 10° C., such as 2 to 8° C.

In a further aspect the pH of said retentate resulting after the diafiltration step iii during storage is adjusted to a pH in the range of pH 4 to pH 6.0, such as pH 4.2 to pH 5.8, such as pH 4.5 to pH 5.6, such as pH 4.5 to pH 5.4, such as pH 4.5 to pH 5.2, such as pH 4.7 to pH 5.3.

The diafiltration step iii may alternatively be performed by way of dialysis. In this case the one or more added salts and water are circulated as a solution on the permeate side of the membrane, and the first retentate, or even the liquid comprising protein, is circulated on the retentate side of the membrane.

Hereby the phenolic compounds may pass the membrane by a passive diffusion process rather than by the active transportation with a forced liquid flow through the membrane. Such diffusion-based process may have the advantage of very low energy input requirements. In an aspect of the dialysis process the dialysis liquid circulated on the permeate side may be substantially pure water and the one or more salts may be added to the first retentate to keep the ionic within a preferred range.

In an aspect of the invention the membrane flux during diafiltration step iii is higher than 5 LMH, such as higher than 6 LMH, such as higher than 7 LMH, such as higher than 8 LMH, such as higher than 9 LMH, such as higher than 10 LMH, such as higher than 11 LMH, such as higher than 12 LMH, such as higher than 13 LMH, such as higher than 14 LMH, such as higher than 15 LMH, such as higher than 16 LMH, such as higher than 17 LMH, such as higher than 18 LMH.

Inactivation of Unwanted Enzymatic Activities

In a further aspect of the invention the proteins isolated according to the invention are treated to obtain an inactivation of enzymatic activities that may be unwanted when the proteins are to be used as food ingredients in certain applications. In particular an inactivation of the esterase and lipolytic activity of patatins and the inactivation of polyphenol oxidase activity is preferred. It is well known that such inactivation may be performed by extensive heating of the proteins, however, such treatment may also lead to uncontrolled polymerization and gelling of the proteins such that the functionality for use in foods is lost or significantly decreased. In our research for inactivation methods without these drawbacks we have surprisingly found that treatment of proteins isolated according to the invention with acidic or alkaline pH values may lead to the preferred inactivation in a controlled manner without losing the functionality of the proteins.

Thus, in an aspect of the invention a solution of the proteins isolated according to the invention are exposed to pH values lower than pH 4.5, such as lower than pH 4.0, such as lower than pH 3.75, such as lower than pH 3.5, such as lower than pH 3.2, such as pH lower than pH 3.0 in a time and temperature interval sufficient to eliminate unwanted enzymatic activity of the proteins. In an aspect of the invention the protein solution is exposed to the low pH comprise a protein with esterase activity to be eliminated. In an aspect of the invention the protein solution exposed to the low pH comprise one or more polyphenol oxidases and the enzymatic activity to be eliminated is the oxidative activity of said polyphenol oxidase. In an aspect of the invention the protein solution exposed to the low pH comprise phospholipase and the enzymatic activity to be eliminated is the phospholipase activity of said phospholipase. In an aspect of the invention the protein solution is exposed to the low pH comprise one or more lipoxygenases and the enzymatic activity to be eliminated is the oxidative activity of said lipoxygenases. In an aspect of the invention the inactivation of enzymatic activity is performed at a temperature in the range of 1-70° C., such as in the range of 1-5° C. such as in the range of 5-60° C., such as in the range of 5-15° C., such as in the range of 10-55° C., such as in the range of 10-15° C., such as in the range of 15-55° C., such as in the range of 18-50° C., such as in the range of 18-48° C., such as in the range of 20-45° C. In an aspect of the invention the inactivation of enzymatic activity is performed in a time span of at least 1 minutes, such as at least 5 minutes, such as at least 10 minutes, such as at least 30 minutes, such as at least 60 minutes, such as at least 120 minutes, such as at least 240 minutes.

Our research has further surprisingly found that a controlled and limited proteolysis of the proteins isolated according to the invention may lead to inactivation of unwanted enzymatic activity without destroying important functional properties, such as the ability to form food gels.

Thus, in an aspect of the invention the inactivation of unwanted enzymatic activity of the protein isolated according to the invention is achieved by treatment of a solution of the protein with one or more proteolytic enzymes. In an aspect of the invention the enzymatic activity of esterases is in part or fully inactivated by the treatment with a protease. In an aspect of the invention the enzymatic activity of phospholipase is in part or fully inactivated by the treatment with a protease. In an aspect of the invention the enzymatic activity of polyphenol oxidase and/or lipoxygenases is in part or fully inactivated by the treatment with a protease.

In an aspect of the invention the one or more proteases are endopeptidase.

In an aspect of the invention the one or more proteases are serine proteases.

In an aspect of the invention the one or more proteases are chosen from the group of trypsin-like, chymotrypsin-like, thrombin-like, elastase-like and subtilisin-like proteases.

In an aspect of the invention the one or more proteases are subtilisin.

Inactivation of Microbial Growth During Processing

Natural protein extracts and other liquids comprising proteins may contain a high level of microbes e.g. naturally originating from the growth and storage of plants in the soil. It is therefore a preferred embodiment of the invention to add a germicidal step during processing of the proteins. This may be applied directly to the liquid comprising proteins, or it may preferably be applied during and/or in between the membrane processing steps according to the invention. Since many proteins are highly sensitive to classical pasteurization by heat treatment leading to loss of functional properties of the proteins it is a preferred embodiment that the germicidal step is performed at a temperature in the range of 2-60° C., such as in the range of 5-55° C., such as in the range of 8-52° C., such as in the range of 10-50° C., such as in the range of 12-45° C., such as in the range of 12-25° C., such as in the range of 12-18° C. In an embodiment of the invention the germicidal step comprise passing of the liquid comprising proteins through a photo bioreactor illuminating high intensity UV light to the liquid. In an embodiment of the invention the UV light has a wavelength primarily in the range of 180-300 nm. In an embodiment of the invention the liquid comprising proteins is illuminated with UV light when passing one or more a spiral-shaped tubes allowing the passage of the UV light. In an embodiment of the invention the inner diameter of said spiral tubes are in the range of 1-15 mm, such as in the range of 2-12 mm, such as in the range of 3-10 mm, such as in the range of 4-9 mm. In an embodiment of the invention the liquid comprising proteins is passed more than one time through the photo bioreactor. In an embodiment of the invention the liquid comprising proteins is passing the photo bioreactor simultaneously with one or more of the membrane filtration steps according to the invention. In an embodiment of the invention the photo bioreactor is inserted into the recirculation loop of one or more of the membrane filtration units applied according to the invention such that the liquid comprising proteins is continuously passing the photo bioreactor. In an embodiment of the invention the photo bioreactor is inserted as a shunt to the recirculation loop of one or more of the membrane filtration units applied according to the invention. In an embodiment of the invention the photo bioreactor is inserted as a recirculation unit to the holding tank of one or more of the membrane filtration units applied according to the invention. In an embodiment of the invention the germicidal step using a photo bioreactor is applied to the protein solution after the membrane filtration steps according to the invention. In an embodiment of the invention the OD600 nm absorption of the protein solution is in the range of 0.02 to 15.0, such as in the range of 0.05 to 8.0, such as in the range of 0.1 to 7.0, such as in the range of 0.5 to 6.0, such as in the range of 0.8 to 5.0, such as in the range of 0.9 to 4.0, such as in the range of 1.0 to 3.0.

In an embodiment of the invention the photo bioreactor is substantially equal to the photo bioreactor disclosed in WO 2019/057257, which is hereby incorporated by reference.

Isolated Compounds, Compositions Comprising the Isolated Compounds and Use and Applications of the Isolated Compounds.

Consumer concerns over highly processed foods and Novel Foods regulations: In some regions and markets there is a desire to minimize the use of highly processed foods and ingredients in the diet. Certain government regulations also demand registration of such highly processed ingredients, which may have a high cost and take several years to perform.

Thus, in embodiments of the invention processing and separation is kept to a minimum to provide still highly functional products with a close to natural composition with only the most toxic and bitter tasting phenolic compounds removed.

In an embodiment of the invention the protein product substantially contains all the proteins present in the liquid comprising proteins

In an embodiment of the invention the protein product substantially contains all the protein and a fraction of the fibers present in the liquid comprising protein.

In an embodiment of the invention the protein product contains all or a fraction of the protein and a fraction of the fibers present in the liquid comprising protein

In an embodiment of the invention the protein product has a true protein content of at least 60%, such as at least 65%, such as at least 70%, such as at least 75%, such as at least 80%, such as at least 85%, such as at least 90%.

In an embodiment of the invention the protein product has a content of insoluble fibers in the range of 2 to 39% on a dry weight basis, such as in the range of 5 to 30%, such as in the range of 7 to 25% on a dry matter basis, such as in the range of 5 to 12%, such as in the range of 5 to 10%, such as in the range of 5 to 8%, such as in the range of 2-5%, such as in the range of 3-8%.

In an embodiment of the invention the proteins and fibers present in the product have been processed as a whole throughout the process without separation and recombination of process streams. Thus, in an aspect of the invention the protein product comprise proteins and fibers that have not been recombined.

In an embodiment of the invention more than 80% of said insoluble fibers in the protein product are small enough to pass a 50 micron filter, such as a 40 micron filter, such as a 30 micron filter, such as a 20 micron filter, such as a 10 micro filter, such as a 5 micron filter, such as a 2 micron filter.

In an embodiment of the invention said insoluble fibers are present in the protein product in a concentration that results in a pellet when the protein product is dissolved/suspended in 0.05 M sodium phosphate pH 6.5 and centrifuged in a tabletop centrifuge at 4000 G for 30 min. In an embodiment of the invention said pellet constitutes in the range of 0.01 to 5 vol/vol % such as 0.02 to 4 vol/vol %, such as 0.03 to 3 vol/vol %, such as 0.05 to 2.8 vol/vol %, such as 0.05 to 2.5 vol/vol %, such as 0.075 to 2.2 vol/vol % relative to the supernatant obtained after centrifugation, when the protein product solution/suspension has a total true protein concentration of 5 mg/ml. In an embodiment of the invention said pellet constitutes in the range of 0.1 to 10 vol/vol %, such as 0.1 to 5 vol/vol %, such as 0.1 to 3 vol/vol %, such as 0.1 to 2 vol/vol %, such as 0.1 to 1 vol/vol %, such as 0.2 to 5 vol/vol %, such as 0.2 to 3 vol/vol %, such as 0.3 to 5 vol/vol %, such as 0.3 to 3 vol/vol %, such as 0.4 to 5 vol/vol %, such as 0.4 to 4 vol/vol %, such as 0.5 to 5 vol/vol %, such as 0.5 to 4 vol/vol %, such as 0.5 to 3 vol/vol %, when the protein product solution/suspension has a total true protein concentration of 5 mg/ml.

The protein products produced according to the invention have surprisingly high functionality and attractive sensory properties when applied as functional protein ingredients in certain food and feed applications.

Thus, in an embodiment of the invention the protein product produced according to the invention is used as a functional ingredient in human food applications or animal feed applications, including pet food applications.

In an embodiment of the invention the protein product produced according to the invention is used as a functional ingredient to obtain a gelling effect in a food or feed preparation. In an embodiment of the invention the gelling effect is obtained at a pH in the range of pH 5.2 to pH 10.0, such as in the range of pH 5.5 to pH 9.5, such as in the range of pH 5.7 to 9.2, such as in the range of pH 5.8 to pH 8.7, such as in the range of pH 5.9 to pH 8.5, such as in the range of pH 6.0 to pH 8.3, such as in the range of pH 6.1 to pH 8.1.

In an embodiment of the invention the protein product produced according to the invention is used as a functional ingredient to obtain an emulsification effect in a food or feed preparation.

In an embodiment of the invention the protein product produced according to the invention is used as a functional ingredient to obtain a foaming and/or a foam stabilisation effect in a food or feed preparation. In an aspect the foam is a feed or food foam, a soap or laundry/detergent foam, a cosmetic foam, a fire-fighting foam, a pollution control foam or a foam for space filling applications.

In an embodiment of the invention the protein product produced according to the invention is used as a functional ingredient to obtain a viscosity enhancing effect in a food or feed preparation

In an embodiment of the invention the protein product produced according to the invention is used as a functional ingredient to obtain a coating and/or encapsulation effect in a food or feed preparation

In an embodiment of the invention the protein product produced according to the invention is used as a functional ingredient to obtain a water binding effect in a food or feed preparation.

In an embodiment of the invention the protein product produced according to the invention is used as a functional ingredient to obtain an enzymatic effect in a food or feed preparation. In an embodiment of the invention the enzymatic effect is an esterase, lipolytic, oxidase, lipoxygenase or protease effect.

In an embodiment of the invention the protein product produced according to the invention is used as a functional ingredient to obtain a protease inhibitor in a food or feed preparation.

1. A method for separation of proteins from first salts and phenolic compounds in a liquid comprising said proteins dissolved in said liquid, said method comprising the steps of:
i. providing a liquid comprising proteins dissolved in said liquid, said liquid further comprising one or more first salts, phenolic compounds and insoluble fibres;
ii. subjecting said liquid to a first cross-flow membrane filtration process wherein at least a portion of the first salts and at least a portion of the phenolic compounds migrate across the membrane into a first permeate and the proteins and said insoluble fibres are retained in a first retentate;
iii. adding water, or one or more second salts and water, to the first retentate, while continuing the membrane filtration process, to create a diafiltrate containing at least a portion of said phenolic compounds and the added second salts.

2. The method according to claim 1, further comprising a germicidal step applied directly to the liquid comprising proteins, or applied during and/or in between the membrane processing steps.

3. The method according to claim 2, wherein the germicidal step is applied to the liquid of step i. and/or the first retentate of step ii.

4. The method according to any one of claim 2 or 3, wherein the germicidal step comprises the exposure of the liquid and/or first retentate to high intensity UV light.

5. The method according to any one of the preceding claims, wherein the germicidal step comprises passing of the liquid comprising proteins through a photo bioreactor illuminating high intensity UV light to said liquid.

6. The method according to any one of the preceding claims, further comprising the step of:
subjecting the first permeate and/or the diafiltrate from said first cross-flow membrane filtration process to a second cross-flow membrane filtration process, whereby at least a portion of the salts present in the first permeate and/or the diafiltrate migrate across the membrane into a second permeate and the phenolic compounds are retained in a second retentate.

7. The method according to any one of the preceding claims, further comprising the step of:
subjecting the first retentate to a microfiltration membrane filtration process, whereby at least a portion of the proteins soluble in said first retentate migrate across the membrane into a microfiltration permeate with a lower turbidity than the turbidity of said first retentate.

8. The method according to any one of the preceding claims, wherein the protein is a protein from plants, yeast or algae, preferably a plant protein.

9. The method according to any one of the preceding claims, wherein the second permeate is used, at least in part, as the source of said water, or one or more second salts and water in claim 1, step iii.

10. The method according to any one of the preceding claims, further comprising the step of adjusting the pH of said liquid to a pH in the range of pH 4.8 to pH 10, such as in the range of pH 4.8 to pH 9.5, such as in the range of pH 4.8 to pH 9.0, such as in the range of pH 5.0 to pH 9.0, such as in the range of pH 5.2 to pH 9.0, such as in the range of pH 5.5 to pH 9.0, prior to said first cross-flow membrane filtration process.

11. The method according to any one of the preceding claims, wherein, when a calcium and/or magnesium salt is added to the retentate prior or during the diafiltration step iii, the pH of the retentate remains within the range of pH 2 to pH 4.5, such as within the range of pH 2.5 to pH 4.2, such as in the range of pH 2.9 to pH 4.0, such as in the range of pH 3.0 to pH 3.7.

12. The method according to any one of the preceding claims, further comprising the step of adjusting the conductivity of said liquid to a conductivity in the range of 2-500 mS/cm2, such as in the range of 1 to 50 mS/cm, such as in the range of 2 to 40 mS/cm, such as in the range of 3 to 30 mS/cm, such as in the range of 3 to 25 mS/cm, such as in the range of 3 to 20 mS/cm, such as in the range of 5 to 17 mS/cm.

13. The method according to any one of the preceding claims, wherein the proteins are selected from the group consisting of protease inhibitors, lipoxygenase, polyphenol oxidase, acid and alkaline phosphatase or mixtures thereof, preferably protease inhibitors.

14. The method according to any one of the preceding claims, wherein the liquid is pretreated e.g. by centrifugation and/or filtration to remove insoluble material of a particle size larger than 10 micron prior to said first cross-flow membrane filtration process.

15. The method according to any one of the preceding claims, wherein said proteins, first salts and said phenolic compounds are in solution in said liquid, and wherein the liquid is preferably water.

16. The method according to any one of the preceding claims, further comprising the step of continuing the addition of said one or more second salts and water to the first retentate while continuing the membrane filtration process until the first retentate contains less than a target amount of the phenolic compounds, whereby the separation of the proteins from phenolic compounds has been achieved.

17. The method according to claim 16, wherein the target amount of remaining phenolic compounds in the first retentate corresponds to less than 5000 mg phenolic compounds per kg protein on the basis of dry weight, such as less than 4000 mg/kg, such as less than 3000 mg/kg, such as less than 2000 mg/kg, such as less than 1500 mg/kg, such as less than 1250 mg/kg, such as less than 1000 mg/kg, such as less than 750 mg/kg, such as less than 500 mg/kg, such as less than 200 mg phenolic compounds/kg protein on the basis of dry weight.

18. The method according to any one of claims 16-17, wherein the amount of phenolic compounds having a molecular weight below 10 kD remaining in the first retentate is less than 1500 mg/kg, such as less than 1000 mg/kg, such as less than 750 mg/kg, such as less than 500 mg/kg, such as less than 300 mg/kg, such as less than 200 mg/kg, such as less than 100 mg/kg, such as less than 50 mg/kg, measured as mg phenolic compounds/kg protein on the basis of dry weight.

19. The method according to any one of claims 7-18, wherein the microfiltration permeate is further treated with a third cross-flow membrane filtration process whereby the protein is concentrated in a third retentate and the salts migrate through the membrane to create a third permeate.

20. The method according to claim 19, wherein the third permeate is used as a diafiltration liquid to be added during the microfiltration process to wash out further proteins from the first retentate into the microfiltration permeate.

21. The method according to any one of the preceding claims, wherein the one or more second salts and water added to the first retentate is an aqueous solution of said second salt(s) in said water.

22. The method according to any one of the preceding claims, wherein the one or more second salts are selected from sodium, potassium, ammonium, calcium or magnesium salts of a mineral acid or an organic acid.

23. The method according to any one of the preceding claims, wherein the one or more second salts are selected from salts of monovalent inorganic anions and cations, such as sodium chloride, potassium chloride, ammonium chloride.

24. The method according to any one of claims 1-23, wherein the one or more salts are selected from sodium sulfate and potassium sulfate.

25. The method according to any one of the preceding claims, wherein the conductivity of the first retentate remains within the range of 1 to 50 mS/cm, such as in the range of 2 to 40 mS/cm, such as in the range of 3 to 30 mS/cm, such as in the range of 3 to 25 mS/cm, such as in the range of 3 to 20 mS/cm, such as in the range of 5 to 17 mS/cm, such as in the range of 6 to 25 mS/cm, such as in the range of 7 to 25 mS/cm, such as in the range of 10 to 25 mS/cm during step ii and step iii.

26. The method according to any one of the preceding claims, wherein the pH of the first retentate remains within the range of pH 2 to pH 10, such as in the range of pH 3.0 to pH 9, such as in the range of pH 3.5 to pH 8.5, such as in the range of pH 4.0 to pH 8.5, such as in the range of pH 4.5 to pH 8.5, such as in the range of pH 5.2 to pH 7.5, preferably in the range of pH 4.2 to pH 6.5, such as in the range of pH 4.5 to pH 6.5, such as in the range of pH 4.8 to pH 6.5, such as in the range of pH 5.0 to pH 6.5 during step ii and step iii.

27. The method according to any one of the preceding claims, wherein the first retentate remains with a temperature in the range of 1 to 60° C., such as 5 to 55° C., such as 10 to 50° C., such as 15 to 48° C., such as 12 to 45° C., such as 18 to 30° C., such as 22 to 28° C. during step ii and step iii

28. The method according to any one of the preceding claims, wherein step iii. comprises adding one or more second salts and water, to the first retentate, while continuing the membrane filtration process, to create a diafiltrate containing at least a portion of said phenolic compounds and the added second salts.

29. The method according to any one of the preceding claims, wherein the addition of the one or more second salts and water in step iii. is performed substantially continuously and at substantially the same flow rate as the first permeate migrates through the membrane.

30. The method according to any one of claims 1-29, wherein the addition of the water, or the one or more second salts and water in step iii is performed in portions, such as portions approximately equal in volume to the volume of the first retentate.

31. The method according to any one of the preceding claims, wherein the total volume of water added during the diafiltration step iii. is in the range of 4 to 20 times the volume of the first retentate, such as 4 to 15 times, such as 5 to 12 times, such as 5 to 10 times, such as 6 to 10 times, such as 7 to 9 times, such as 5 to 9 times the volume of the first retentate.

32. The method according to any one of the preceding claims, wherein step iii. comprises a second phase of diafiltration following the addition of one or more salts and water wherein the diafiltration is continued with the addition of water without adding any further salts.

33. The method according to any one of the preceding claims, wherein, step iii is continued with water and without the addition of further salts until the conductivity of the retentate is less than 10 mS/cm, such as less than 8 mS/cm, such as less than 5 mS/cm, such as less than 4 mS/cm, such as less than 3 mS/cm, such as less than 2 mS/cm, such as less than 1 mS/cm.

34. A protein product, comprising protein and a fraction of the insoluble fibers present in the liquid comprising proteins used for the production of the protein product wherein the content of said insoluble fibers in the product is in the range of 2 to 39% on a dry weight basis and wherein the amount of phenolic compounds in the protein product corresponds to less than 2000 mg/kg protein on the basis of dry weight.

35. A protein product according to claim 34 wherein the number of viable bacteria or fungal cells is lower than 100 000 per g dry matter, such as lower than 10 000 per g dry matter, such as lower than 5000 per g dry matter, such as lower than 1000 per g dry matter.

36. A protein product according to any one of claims 34-35, wherein the product has a true protein content of at least 60%, such as at least 65%, such as at least 70%, such as at least 75%, such as at least 80%, such as at least 85%, such as at least 90%.

37. A protein product according to any one of claims 34-36 wherein the content of insoluble fibers is in the range of 5 to 30%, such as in the range of 7 to 25%, such as in the range of 5 to 12%, such as in the range of 5 to 10%, such as in the range of 5 to 8%, such as in the range of 2-5%, such as in the range of 3-8% on a dry weight basis.

38. A protein product according to claims 34-37 wherein the proteins and fibers have not been non-recombined.

39. A protein product according to claims 34-38 wherein more than 80% of said insoluble fibers in the product are small enough to pass a 50 micron filter, such as a 40 micron filter, such as a 30 micron filter, such as a 20 micron filter, such as a 10 micro filter, such as a 5 micron filter, such as a 2 micron filter.

40. A protein product according to claims 34-39, wherein the amount of phenolic compounds in the protein product corresponds to less than 1500 mg/kg, such as less than 1250 mg/kg, such as less than 1000 mg/kg, such as less than 750 mg/kg, such as less than 500 mg/kg, such as less than 200 mg phenolic compounds/kg protein on the basis of dry weight.

41. The protein product according to any one of claims 34-40, having a water content of less than 10%, such as less than 7%, such as less than 6%, such as less than 5%, such as less than 4%.

42. The protein product according to any one of claims 34-41, being an aqueous suspension or a paste of said protein and fibers having a dry matter content of 0.1% to 80%, such as 0.5% to 75%, such as 1% to 73%, such as 2% to 65%, such as 5% to 60%, such as 10% to 50%, such as 10% to 40%, such as 10% to 30%, such as 15% to 28%.
